(19) United States
(12) Patent Application Publication
(10) Pub. No.: US 2010/0030235 A1
(43) Pub. Date: Feb. 4, 2010

(54) APPARATUS FOR REMOVING TICKS

(75) Inventor: Lionel Bloom, Kings Park (AU)

Correspondence Address: EDWARDS ANGELL PALMER & DODGE LLP, P.O. BOX 55874, BOSTON, MA, 02205, US

(73) Assignee: Veterinary Companies of Australia Pty Ltd, Kings Park New South Wales (AU)

(21) Appl. No.: 11/630,235

(22) PCT Filed: Jun. 20, 2005
(86) PCT No.: PCT/AU2005/000885

§ 371 Date: Sep. 21, 2009

(30) Foreign Application Priority Data
Jun. 21, 2004 (AU) 2004100487
Jun. 21, 2004 (AU) 2004903331

(51) Int. Cl. A61B 17/50

(52) U.S. Cl. 606/131

(57) ABSTRACT

An apparatus (1) for removing ticks from animals and humans, comprising an elongated grip (2) having a longitudinal axis; a first bifurcated tip (3) located at one end of the elongated grip; and a second bifurcated tip (4) located at a second end of the elongated grip, wherein in the first and second bifurcated tips extend substantially perpendicular to the longitudinal axis of the elongated grip. The first and second bifurcated tips are preferably curved towards the elongated grip and extend from the longitudinal axis of the elongated grip in opposing directions.

### FIELD OF THE INVENTION

This invention relates to an improved apparatus for removing ticks and the like from the skin of animals and humans.

### BACKGROUND OF THE INVENTION

Ticks are very hazardous to the health of animals and humans. They infect the victim by penetrating the epidermis with their rostrum and thus remain adhered to the skin until forcibly removed. Ticks often carry bacteria which may be passed to the animal or human whilst the tick is engorging itself on the carriers blood. In humans this can lead to paralysis, allergic reactions and in some cases Lyme Disease (a disorder having severe flu like symptoms). In animals, tick infestations can lead to paralysis and in severe cases death.

Traditionally ticks are removed using tweezers, burning them off and/or application of acaricide preparation. Tweezers have the advantage in that they can be used for various sized ticks. However they can result in the body of the tick being squashed and host being covered in blood and saliva.

Australian patent 695632, describes an implement having an elongated handle with a perpendicular forked region. The forked region is fitted around the head and thorax of the tick, the handle of the implement rotated until the tick is removed from the skin. A number of implements with differing sized forked regions are required to be utilised for the removal of ticks of varying sizes.

### OBJECT OF THE INVENTION

It is an object of the invention to overcome or alleviate one or more of the above disadvantages or to provide the consumer with a useful or commercial choice.

### SUMMARY OF THE INVENTION

In one form, although not necessarily the broadest or only form, the invention resides in an apparatus, for removing ticks from animals and humans, comprising:

an elongated grip having a longitudinal axis;

a first bifurcated tip located at one end of the elongated grip; and

a second bifurcated tip located at a second end of the elongated grip,

wherein in the first and second bifurcated tips extending substantially perpendicular to the longitudinal axis of the elongated grip.

The elongated grip is preferably cylindrical in nature. The surface of the elongated grip may be contoured or textured to facilitate gripping in the users hand.

The first and second bifurcated tips may be rectilinear or curved towards the elongated grip. The first and second bifurcated tips may extend substantially perpendicular from the longitudinal axis of the elongated grip in the same or opposing directions. The bifurcated tips are preferably chamfered at their extremity. The tips are bifurcated to form prongs. The space between the prongs preferably forms an elongated V shape adapted to releasably engage a tick for removal. Preferably, the second bifurcated tip is smaller than the first bifurcated tip.

### BRIEF DESCRIPTION OF THE DRAWINGS

Embodiments of the invention will be described, by way of example only, with reference to the accompanying drawings in which:

FIG. 1 is a schematic representation of an embodiment of the apparatus of the invention.

### DETAILED DESCRIPTION

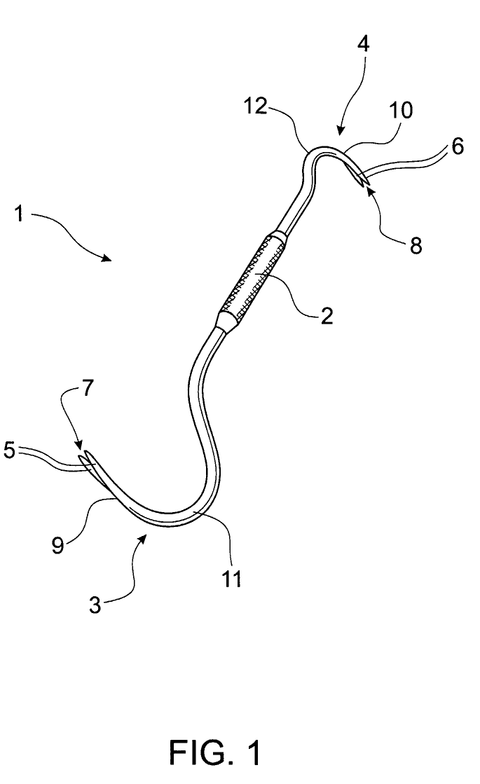

FIG. 1 shows an apparatus or tick remover 1 having an elongated grip 2. In this embodiment the elongated grip 2 is cylindrical in nature and has a fine stipple finish. It will be appreciated that the elongated grip 2 may have a range of surface textures to facilitate gripping, including smooth, rippled, striated and the like. Furthermore, the elongated grip 2 may be contoured; cylindrical; and/or be square, hexagonal or octagonal in cross-section to facilitate gripping by the user's hand.

At one end of the elongated grip 2 extends a first bifurcated tip 3. At a second end of the elongated grip 2 is located a second bifurcated tip 4. The bifurcated tips 3 and 4 have two prongs each, 5 and 6 respectively. The leading edge of the prongs 5 and 6 is chamfered. The prongs 5 and 6 define an elongated V-shaped space 7 and 8, respectively. The bifurcated tips 3 and 4 have an outer surface 9 and 10, respectively, which is substantially flat to allow for sufficient contact with the skin of the infected animal or human.

In this embodiment the first and second bifurcated tips 3 and 4 extend from the elongated grip 2 in a curved manner, such as a goose-neck shape, shown as 11 and 12, respectively in FIG. 1, and extend in opposite directions from the longitudinal axis of the elongated grip 2. It will be appreciated that the curved sections 11 and 12 may be replaced with linear or angled sections. Furthermore, the curved sections 11 and 12 may extend in the same direction from the longitudinal axis of the elongated grip 2.

The first bifurcated tip 3 in this embodiment is shown as being larger in overall size and depth of the elongated V-shaped space 7 than the second bifurcated tip 4 and its related elongated V-shaped space 8, to allow for the tick remover 1 to be readily used in the removal of different sized ticks. It will be appreciated that the first and second bifurcated tips, 3 and 4, may be the same size and have the same or different depth of their respective elongated V-shaped space, 7 and 8.

In use the tick remover 1 is held in the hand of the user by the elongated grip 2 and either the first or second bifurcated tip (3 or 4) nears the tick located in the skin of an animal or human with the pair of prongs (5 or 6) of the first or second bifurcated tip (3 or 4) directed towards the tick. The prongs (5 or 6) of the tip are slid underneath the tick to result in the tick being located firmly between the prongs (5 or 6). The user then applies a slight pulling force away from the skin prior to rotating or twisting the apparatus between the thumb and fore index finger until the tick loosens. The ticks generally loosen or are removed with the second or third twist of the apparatus. If preferred the user may then apply antiseptic.

Ticks may vary significantly in size through their stages of development from larvae, pupa or adults and with the variety of tick species. Furthermore a tick may become significantly engorged with blood if it has been located on the skin for an extended period of time. The tick removal apparatus of the invention provides an effective way of removing ticks from the skin of animals or humans regardless of the size of the tick to be removed without the inconvenience of having multiple sized tools, which may be easily lost or make removing many ticks from an infected animal a very time consuming exercise, or risking squashing the ticks during removal.

It should be appreciated that various other changes and modifications may be made to the invention described without departing from the spirit or scope of the invention.

1. An apparatus, for removing ticks from animals and humans, comprising:
an elongated grip having a longitudinal axis;
a first bifurcated tip located at one end of the elongated grip; and
a second bifurcated tip located at a second end of the elongated grip,
wherein in the first and second bifurcated tips extending substantially perpendicular to the longitudinal axis of the elongated grip.

2. The apparatus of claim 1, wherein the first and second bifurcated tips are curved towards the elongated grip.

3. The apparatus of claim 1, wherein the first and second bifurcated tips are rectilinear to the elongated grip.

4. The apparatus of claim 1, wherein the first and second bifurcated tips extend substantially perpendicular from the longitudinal axis of the elongated grip in opposing directions.

5. The apparatus of claim 1, wherein the first and second bifurcated tips extend substantially perpendicular from the longitudinal axis of the elongated grip in the same direction.

6. The apparatus of claim 1, wherein the second bifurcated tip is smaller than the first bifurcated tip.

7. The apparatus of claim 1, wherein the first and second bifurcated tips are bifurcated to form prongs, which form an elongated V-shaped space between the prongs adapted to releasably engage a tick for removal.

8. The apparatus of claim 1, wherein the bifurcated tips are chamfered at their extremities.
